(19) United States
(12) Patent Application Publication
(10) Pub. No.: US 2025/0032706 A1
(43) Pub. Date: Jan. 30, 2025

(54) Medical Safety Needle Assemby

(71) Applicant: Design 33 Inc., Cheyenne, WY (US)

(72) Inventor: John Simon Forber, Liguge (FR)

(73) Assignee: Design 33 Inc., Cheyenne, WY (US)

(21) Appl. No.: 18/716,120

(22) PCT Filed: Dec. 5, 2022
(86) PCT No.: PCT/IB2022/000681

§ 371 Date: Jun. 3, 2024

(30) Foreign Application Priority Data
Dec. 3, 2021 (FR) FR2112969

(51) Int. Cl. A61M 5/158; A61M 39/02

(52) U.S. Cl. CPC A61M 5/158; A61M 2039/0229

(57) ABSTRACT

Medical safety needle assembly comprising a medical needle having a distal and a proximal end and a stem extending there between, the needle having a tip at its distal end, the proximal end of the needle being connectable to a fluid path, and the needle stem having a 80°-90° bend, and a safety device comprising a base plate and a housing the base plate and the housing enclosing together a cavity, the safety device being slidably arranged on the needle stem distal from the bend, and the needle stem passing through a distal opening in the base plate and a proximal opening in the housing, the needle having an inserted position with the needle tip extending in distal direction from the base plate and a retracted position with the needle tip being in a position inside the cavity, characterized in that the housing consists of a first part attached to the base plate and a second part (32) attached by means of extendible attachment elements (33) to the first part, wherein the proximal opening is arranged in the second part (32) of the housing, and wherein the attachment elements (33) allow a movement of the second part (32) of the housing relative to the first part in the proximal direction of the needle stem between a first and a second position; the needle stem comprises a dead stop arranged distal from the proximal opening in the second part (32) of the housing that cannot pass through the opening.

The present invention relates to a medical safety needle assembly.

Implantable vascular access systems are used extensively in the medical field to facilitate the performance of repeated injections of therapeutic treatments inside the body of a patient. The vascular access system is generally comprised of a vascular access port that is attached to a catheter. The vascular access port generally consists of a housing with an elastomeric septum exposed on the upper surface. Beneath the elastomeric septum is a reservoir that can be accessed by the tip of a needle when it passes the elastomeric septum. The vascular access port has a tubular elongated member extending from the reservoir that allows attachment of the catheter.

The vascular access system is implanted into the body of a patient. The distal tip of the catheter is disposed at a predetermined location where the therapeutic agent is delivered, generally the entrance of the right atrium just beyond the superior vena cava. Once the vascular access system is implanted, the care giver can insert the tip of the needle through the skin, the elastomeric septum and into the reservoir space to deliver the treatment. In order to minimize the height of the needle extending from the skin, the needles are bent approximately 90° so that they extend minimally above the skin of the patient.

The tip of a preferred needle type for this kind of application has a bend close to the principle bevel of the needle. This bend is such that once formed the principle bevel is approximately parallel to the axis of the unbent portion of the needle stem. The generally accepted terminology for this type of needle tip is a “Huber” tip. When a needle of this conception is forced through the elastomeric septum, it creates just a slit in the septum. When the needle is removed, the slit surfaces return in contact and under the radial compression of the septum and the intrinsic characteristics of the elastomeric material, a seal is maintained. If a regular hypodermic needle was inserted into the elastomeric septum there is an elevated risk that the needle may core particles from the septum. This could potentially impair the sepium and/or liberate the particles that could obstruct the catheter or be released into the vasculature of the patient.

As with any needle type device, there exists the risk of inadvertent needle sticks, that generally occur when the needle is removed from the patient prior to disposal of the needle. This risk is particularly significant in the case of Huber needles being removed from a vascular access system. This is because the force to remove a Huber needle from a port is nigh due the fact that the needle is being compressed radially by the elastomeric septum of the vascular access port. Also the care giver generally needs to stabilize the vascular access port with the fingers of the opposite hand at a location close to the exit site of the needle.

To address the problem of inadvertent needle sticks, various safety devices have been designed. One safety device is described in U.S. Pat. No. 5,755,694 that discloses a needle base disposed over a segment of the needle at its proximal end, comprising two generally flat wings made of a flexible plastic material, with one hinge connecting each of the wings to the needle base. Upon removal of the needle from the patient, the wings flex against the moveable members that keeps them adjacent to the needle until the needle is completely removed from the skin of the patient after which the wings surround the needle to prevent the tip from making contact outside the needle base.

Many advancements have been made in needle devices for use with vascular access ports to reduce the risk of needle sticks.

However, there is an additional problem with many safety devices. In many cases, the cover and mechanism is very large. The larger the device is above the skin, the higher the risk that the needle might be inadvertently hit and become dislodged from the patient. Also the fact that the device extends significantly above the skin of the patient is cosmetically undesirable.

Accordingly, the object of the present invention is to provide a low profile medical safety needle assembly.

The object of the invention is achieved with a medical safety needle assembly comprising a medical needle having a distal and a proximal end and a stem extending there between. The needle has a tip at its distal end, and the proximal end of the needle is connectable to a fluid path like e.g., a tubing with a connection hub for connecting the tubing to a syringe. The needle stem has a 80°-90° bend. The medical safety needle assembly comprises further a safety device comprising a base plate and a housing. The base plate and the housing enclose together a cavity. The safety device is slidably arranged on the needle stem distal from the bend, and the needle stem passes through a distal opening in the base plate and a proximal opening in the housing. The needle has an inserted position with the needle tip extending in distal direction from the base plate and a retracted position with the needle tip being in a position inside the cavity.

The needle assembly in accordance with the invention is characterized in that the housing consists of a first part attached to the base plate and a second part attached by means of extendible attachment elements to the first part, wherein the proximal opening is arranged in the second part of the housing and wherein the attachment elements allow a movement of the second part of the housing relative to the first part in the proximal direction of the needle stem between a first and a second position. Furthermore, the needle stem comprises a dead stop arranged distal from the proximal opening in the second part of the housing that cannot pass through the opening.

The design of the housing allows a very low profile as long as the needle is inserted in a patient. However, such a low profile will normally not allow for a sufficiently large cavity to fully enclose the needle tip. The needle assembly has to have a safety mechanism that encloses the needle tip when the needle is retracted. The needle must be prevented from leaving the cavity in the housing of the base plate in both directions, distally and proximally.

The dead stop of the needle stem is used to prevent the needle from leaving the safety device in proximal direction. The dead stop can for example be a deformation in diameter of the needle stem or the bend of the needle tip of a Huber type needle or a combination thereof. The proximal opening of the safety device is sized to let the needle stem pass through it smoothly, whereas the dead stop is blocked in the opening.

The efficiency of the dead stop may be enhanced by using a retention ferrule that slides on the shaft but cannot pass over the dead stop. Such a retention ferrule may for example have a circumferential collar with a diameter that cannot pass through the proximal opening of the housing. The retention ferrule may also have a collar that extends in the direction of the needle stem and prevents the retention ferrule from passing over the bend of a Huber type needle close to its tip whereas it can slide on the rest of the needle stem proximal from the bend.

When the dead stop of the needle stems blocked in the opening of the second part of the housing of a safety device in accordance with the present invention, the latter may be lifted upwards. The extendible attachment elements secure the second part of the housing in its first position to obtain the required low profile as long as no lifting force is exercised on it. Thus, as long as the needle is inserted, the safety device has a very low profile.

Once the needle is withdrawn, the dead stop of the needle stem is blocked in the opening in the second part of the housing. When the needle is further drawn back, the second part of the housing is pulled upwards in proximal direction of the needle. The extendible attachment elements between the two parts of the housing allow the movement of the second part of the housing until its defined second position. Because the second part of the housing is now in its second, upper position, the cavity inside the housing has grown in height, and the cavity can accommodate the whole needle tip from the Huber bend to the tip.

The attachment elements may connect the first and the second part of the housing in any way that allows the second part to move relative to the first part. In one embodiment, the attachment elements may be flexible. They may for example be S- or Z-shaped when the second part of the housing is in its first position and may be expanded to be straight in the second position. In such configuration, the attachment elements may retain the second part of the housing elastically in the first position, and a slight force is necessary to expand the attachment elements. In the straight configuration, they may keep the second part of the housing elastically in the second position and it may not return into the first position, or at least a considerable force would be necessary to achieve this.

The movement of the second part of the housing may be guided by guiding elements that may be comprised in the housing or be attached to the base plate.

In one embodiment, the second housing part may consist of several telescopic elements. This allows a particular low profile shape of the assembly in the inserted position of the needle but allows at the same time to form a cavity that is large enough to securely accommodate the entire needle tip.

The internal distance between the second housing element in its second position and the base plate is sufficient to let the needle tip pass through the opening in the base plate so that it is contained in the cavity of the housing.

Preferably, the base plate may comprise a safety mechanism for closing the distal opening once the needle tip has passed the base plate.

Such safety mechanism may for example comprise a spring leaf that is laterally biased in contact with the needle stem and is provided with a sheet, for example a metal sheet that is positioned orthogonally to the direction of the needle stem and that closes the opening in the base plate once the needle tip passes the opening. In this design, the spring leaf may be slightly biased and apply a low lateral force on the needle stem that is low enough to allow the needle to slide through base plate and housing without significant effort. Once the needle tip has passed the opening in the base plate, the leaf spring swings into an unbiased position and closes the opening in the base plate with the sheet. The needle tip is thus fully enclosed in a cavity formed by the housing together with the base plate.

In a preferred embodiment, a handle may be attached to the needle to insert and to remove it. The handle may be movable between a first position parallel to the base plate and a second position orthogonal to the base plate. In this way, a low profile may be achieved when the handle is in its position parallel to the base plate. It may be brought into a position orthogonal to the base plate to insert or to retract the needle from the patient.

Various embodiments of the invention are described in greater detail in the following, using the attached figures:

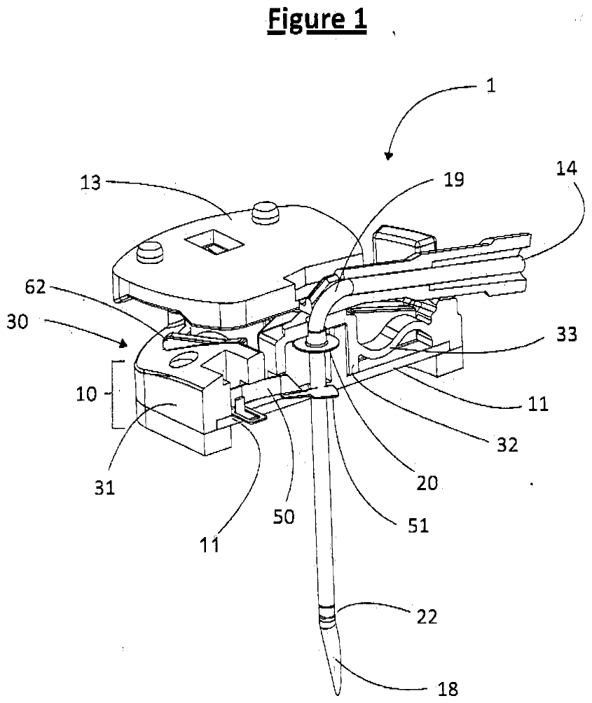

FIG. 1 is an isometric view of a cross section of one embodiment of the present invention in the inserted needle position;

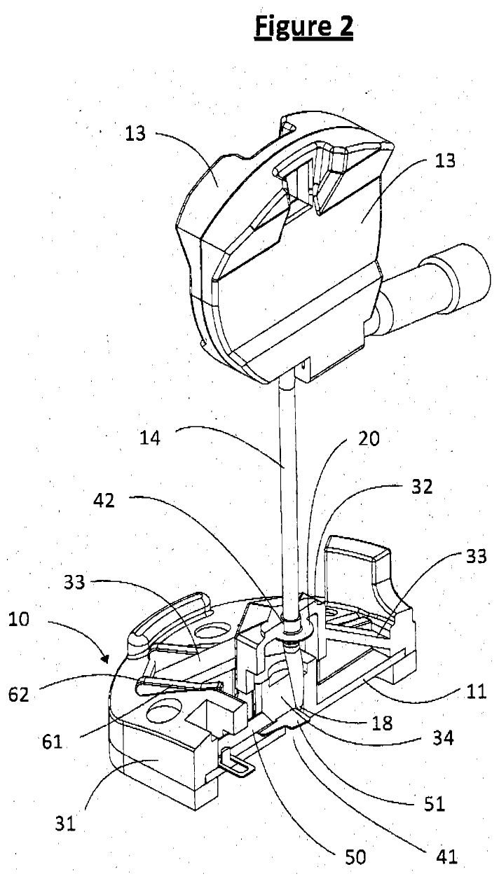

FIG. 2 is an isometric view of a cross section of the embodiment of FIG. 1 in the retracted needle position;

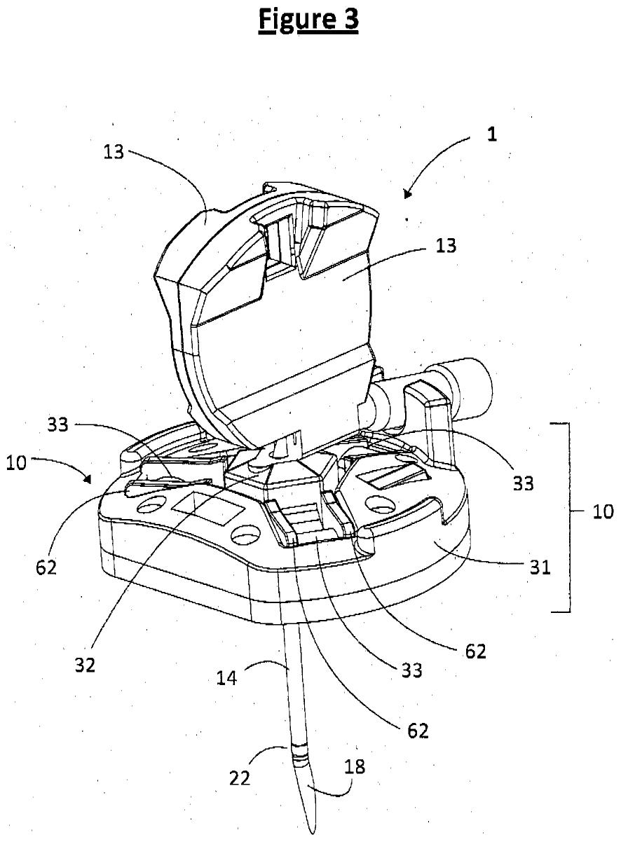

FIG. 3 shows an isometric view of the embodiment of FIGS. 1 and 2 in the inserted needle position with the handle folded up to retract or insert the needle;

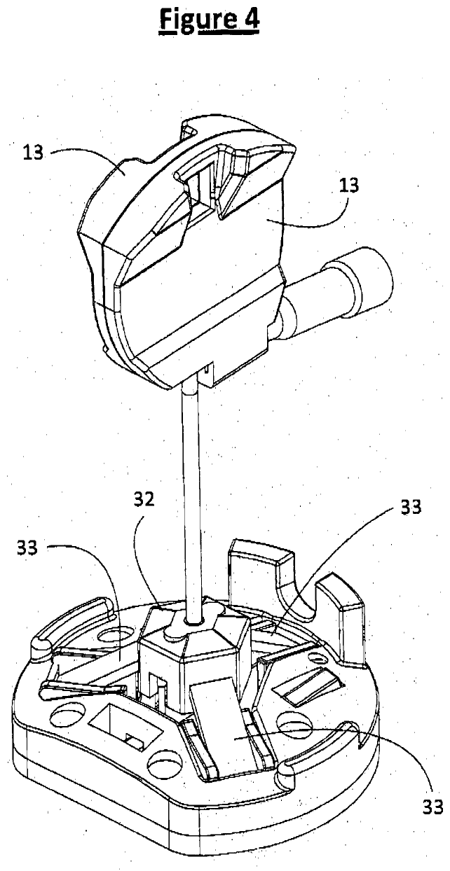

FIG. 4 is an isometric view of the assembly of FIG. 3 in the retracted needle position;

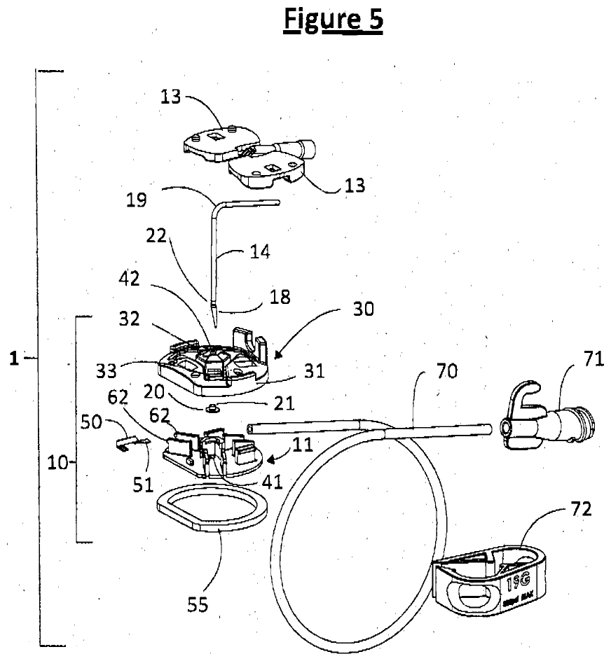

FIG. 5 is an exploded view of the embodiment of the previous figures that shows the needle safety assembly together with additional, optional elements;

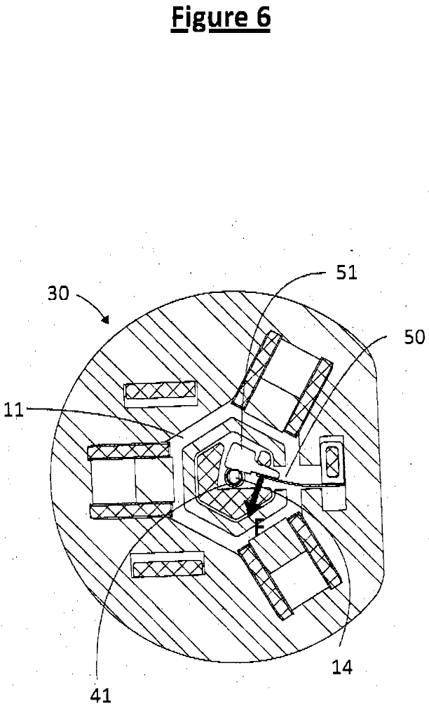

FIG. 6 is a top view of an embodiment of a closure mechanism for the needle passage in the base plate.

It will be further understood that the terms “comprises” and/or “comprising” when used in this specification, specify the presence of stated features, steps, operations, elements and/or components, but do not preclude the presence or addition of one or more other features, steps, operations, elements, components, and/or groups thereof.

FIG. 1 is an isometric view of a cross section of one embodiment of the medical safety needle assembly 1 in accordance with the present invention in the inserted needle position. The assembly comprises a safety device 10 comprising a base plate 11, a handle 13 comprising two parts (only one part shown in this figure), and a needle 14 with a Huber tip 18 at its distal end. The proximal end of the needle is connectable to a fluid path like a tubing.

The safety device 10 of the assembly 1 comprises further a housing 30 that consists of a first part 31 attached to the base plate 11 and a second part 32 attached by means of attachment elements 33 to the first part 31. The needle traverses the first and the second part of the housing wherein a distal opening 41 is provided in the base plate 11 and a proximal opening 42 is provided in the second part of the housing 32.

The attachment elements 33 are extendible and allow a movement of the second part 32 of the housing relative to the first part 31 in the proximal direction of the needle stem between a first and a second position.

The needle 14 extends slidably through the distal opening 41 in the base plate 11 and through the proximal opening 42 in the second part 32 of the housing. In the shown inserted position, the needle tip extends in distal direction from the base plate 11. The needle stem has a 80°-90° bend 19 at its section proximal of the safety device 10.

A retention ferrule 20 is arranged on the needle stem between the Huber tip 18 and the needle bend 19 inside the housing. The retention ferrule may not s ide over the dead stop 22 that is formed by the slight needle bend of the Huber tip 18. In addition, a deformation in the stem diameter may be located on the needle stem close to the Huber 22 bend to ensure that the retention ferrule does not slide further down towards the needle tip.

A spring leaf 50 is laterally biased in contact with the needle stem and is provided with a metal sheet 51 that is positioned orthogonally to the direction of the needle stem. The spring leaf is biases towards the needle stem so that it closes the opening 41 in the base plate 11 once the needle tip has passed it.

The handle 13 is shown in its position parallel to the base plate 11. The assembly has a particular low profile in the shown configuration when the needle is inserted into the patient.

The risk that the needle might be inadvertently hit and become dislodged from the patient is consequently low. Also, the cosmetic appearance of the device is discreet.

FIG. 2 shows the embodiment of FIG. 1 in the retracted needle position. For retracting the needle, the two parts of the handle 13 are turned into a position orthogonal to the base plate 11 so that a care giver can pull the needle 14 upwards. The needle slides through the distal and proximal openings 41, 42 in the base plate 11 and the second part 32 of the housing. The retention ferrule 20 slides on the needle stem until it reaches the bend 22 of the Huber tip 18 that acts as a dead lock and is then blocked on the needle stem by the bend 22 or an additional deformation in diameter of the needle stem in this area. When the retention ferrule 20 that is blocked on the needle stem comes into contact with the second part of the housing 32 it lifts this part upwards. The flexible and extendible attachment elements 33 are transformed from the original s-shaped form into a straight form and limit the upward movement of the second housing part 32. The device of this embodiment comprises three flexible attachment elements 33 in a star-shaped arrangement, whereas two of them are represented in this figure. The third is not shown due to the sectional cut representation.

When the second housing part 32 is moved upwards by the needle, the height of the cavity 34 inside the housing increases so that the whole Huber tip 18 of the needle may be enclosed in the cavity 34.

The design of the safety device 10 as described allows for a low profile of the device in the inserted position. The size of the cavity inside the housing will nevertheless be sufficient to entirely enclose the Huber needle tip in the retracted position when a low profile is no longer of importance.

Once the needle tip 18 has passed the opening 41 in the base plate 11, the spring leaf swings into a position where the metal sheet 51 that is fixed to the spring leaf in a position orthogonal to the direction of the needle shaft closes the opening 41 in the base plate 11.

FIG. 3 shows the safety needle assembly 1 in the inserted needle position in isometric view where the two parts of the handle 13 are folded up so that a care giver can easily insert or retract the needle. The folded up handle 13 allows the view on the second part 32 of the housing and the flexible attachment elements 33 that are s-shaped as long as the second housing part 32 is in its first, lower position.

FIG. 4 shows the embodiment of FIG. 3 with the needle being retracted. The needle has pulled the second housing part 32 upwards, so that the attachment elements 33 are now in their expanded, straight form and limit the upward movement of second housing part 32.

FIG. 5 shows a medical safety needle assembly 1 in an exploded view in a set that may be commercialized in this configuration. The Huber tip needle 14 shows two bends, one slight bend at the Huber tip 18 of the needle and one 80-90° bend 19 further in proximal direction. The needle stem between the first and the second bend allow the needle to slide through the openings 41 and 42 in the base plate 11 and in the second part 32 of the housing 30 of the safety device 10.

The housing 30 comprises a first part 31 and a second part 32 that are attached to each other by means of flexible, extendible attachment elements 33 that are initially s-shaped and allow a position of the second housing part 32 in the middle of the first housing part 31 without significantly protruding therefrom. The second housing part 32 has a proximal opening 42 to slidably accommodate the needle shaft between the two bends.

The safety device 10 of the needle assembly 1 further comprises a base plate 11 with a distal opening 41. The base plate may comprise a circumferential distance element 55 that may be integral with or connected to the base plate 11. It may in particular be a foam ring. The housing part 31 and the base plate 11 may be connected with each other by means of connection elements 60. The base plate 11 also has guiding elements 61 for guiding the second part of the housing from its first into its second positon. Additional retention elements 62 resist the tension applied to the housing part 31 when the extendible attachment elements 33 are extended to their straight configuration.

The safety device 10 further comprises a spring leaf 50 that is in its assembled position in lateral and biased contact with the needle shaft. It has a lateral metal sheet 51 that is arranged orthogonally to the direction of the needle shaft traversing the proximal and distal openings 42 and 41 in the housing and the base plate. The sheet closes the opening 41 in the base plate when the needle is retracted and no longer passes through the opening of the base plate.

The retention ferrule 20 may be put on the needle stem in the section between the first and the second bend of the needle. The height of the collar 21 of the retention ferrule does not allow the ferrule to slide over the bend at the tip of the needle so that the retention ferrule will be blocked in this position when the needle is drawn upwards and will pull the second housing part 32 upwards from its first into its second position when the needle is withdrawn from the patient.

The shown safety needle assembly comprises in addition a handle 13 that will be attached to the needle on the proximal side of the second bend 19, but close to the bend. The handle comprises two flaps that may folded up to form a single handle in an essentially orthogonal position to the base plate and housing so that the needle 14 may be precisely inserted or withdrawn.

The proximal end of the needle may be connected to a tubing 70 that may in turn have a connection hub 71 at its proximal end, for connecting for example a syringe. The connection hub may have a standard Luer connection for this purpose or any other suitable connection element. A clamp 72 may be added to the set to limit or stop the fluid path through the tubing 70.

FIG. 6 shows the base plate 11 with the spring leaf 50 that acts as safety mechanism for closing the needle opening 41 in the base plate 11. As long as the needle 14 passes through the needle opening 41, the spring leaf is in lateral contact with the needle stem and slightly biased, as indicated by the arrow F. When the needle is withdrawn and no longer extends through the needle opening 41, the spring leaf will move in the direction of arrow F, and the metal plate 51 will close the needle opening 41, so that the needle tip may no longer leave the cavity that is formed by the base plate 11 and housing 30.

What is claimed is:

1. Medical safety needle assembly (1) comprising:
a medical needle (14) having a distal and a proximal end and a stem extending there between, the needle having a tip (18) at its distal end, the proximal end of the needle being connectable to a fluid path, and the needle stem having a 80°-90° bend (19),
and a safety device comprising a base plate (11) and a housing (30), the base plate and the housing enclosing together a cavity, the safety device being slidably arranged on the needle stem distal from the bend (19), and
the needle stem passing through a distal opening in the base plate and a proximal opening in the housing,
the needle (14) having an inserted position with the needle tip extending in distal direction from the base plate (11) and a retracted position with the needle tip being in a position inside the cavity,
characterized in that the housing (30) consists of a first part (31) attached to the base plate (11) and
a second part (32) attached by means of extendible attachment elements (33) to the first part (31) wherein the proximal opening is arranged in the second part (32) of the housing, and wherein the attachment elements (33) allow a movement of the second part (32) of the housing relative to the first part (31) in the proximal direction of the needle stem between a first and a second position;
the needle stem comprises a dead stop (22) arranged distal from the proximal opening in the second part (32) of the housing that cannot pass through the opening.
